(19) United States
(12) Patent Application Publication
(10) Pub. No.: US 2013/0306917 A1
(43) Pub. Date: Nov. 21, 2013

(54) QUINONE COMPOUNDS FOR USE IN PHOTOVOLTAIC APPLICATION

(71) Applicants: Floryan Decampo, Shanghai (CN); Gerard Mignani, Lyon (FR); Bertrand Pavageau, Villenave D'ornon (FR)

(72) Inventors: Floryan Decampo, Shanghai (CN); Gerard Mignani, Lyon (FR); Bertrand Pavageau, Villenave D'ornon (FR)

(73) Assignee: RHODIA OPERATIONS, AUBERVILLIERS (FR)

(21) Appl. No.: 13/880,283

(22) PCT Filed: Oct. 21, 2011
(86) PCT No.: PCT/EP11/05442

§ 371 Date: Aug. 2, 2013

(30) Foreign Application Priority Data
Oct. 21, 2010 (FR) 1058623

(51) Int. Cl. H01L 51/00

(52) U.S. Cl. CPC H01L 51/0032; H01L 51/0083; H01L 51/0035; H01L 51/0036
USPC 252/519.21; 549/75; 549/59; 548/444; 558/410; 556/144; 549/472; 548/416; 549/71; 252/500; 528/266; 528/265

(57) ABSTRACT

The invention relates to a photovoltaic coating containing a mixture of organic N-type (acceptor) and P-type (donor) semiconductor compounds, which makes it possible, when selecting the donor/acceptor pair, to modulate the semiconductor properties of the photovoltaic coating so as to enable the use thereof within a photovoltaic device, wherein one of the organic semiconductors includes a quinone core.

The present invention relates to the field of photovoltaic devices, so-called third generation devices which apply organic semi-conductors.

Such devices (in particular photovoltaic cells) which, in order to ensure a photovoltaic effect, apply organic semi-conductors (often designated by OSC, (Organic Semi-Conductors)), are of a recent design. These systems, which began to be developed during the 1990's, aim at being substituted in the long run to first and second generation devices, which apply inorganic semi-conductors.

In these photovoltaic devices which apply OSCs, the photovoltaic effect is ensured by the joint application of two distinct organic compounds used as a mixture, i.e.:
a first organic compound having a P type (electron donor) semi-conductor nature, which is generally a compound, preferably polymeric, which has electrons engaged in advantageously delocalized, pi bonds, and which is most often a conjugate polymer; and
a second organic compound, which is not miscible with the first compound under the conditions of use of the photovoltaic device, and which has a semi-conductor nature of the N type (electron acceptor).

The photovoltaic effect is obtained by placing both organic semi-conductors between two electrodes, in the form of a coating comprising both of these semi-conductors as a mixture (this coating being in direct contact with both electrodes, or optionally connected to at least one of the electrodes via an additional coating, for example a charge collecting coating); and by irradiating the thereby achieved photovoltaic cell with adequate electromagnetic radiation, typically with light from the solar spectrum. To do this, one of the electrodes is generally transparent to the electromagnetic radiation used: in a way known per se, a transparent anode in ITO (indium oxide doped with tin) may notably be used. Obtaining the coating based on the mixture of both organic semi-conducting compounds between the electrodes is typically achieved by depositing a solution of both compounds in a suitable solvent and then by evaporating this solvent.

Under the effect of the irradiation, the electrons of the P type organic semi-conductor are excited, typically according to a so-called π-π* transition mechanism (passing from the highest occupied molecular orbital (HOMO) to the lowest unoccupied molecular orbital (LUMO)), which leads to an effect similar to the injection of an electron from the valency band into the conduction band in an inorganic semi-conductor, which leads to the creation of an exciton (electron/hole pair).

Because of the presence of the N type organic semi-conductor in contact with the P type semi-conductor, the thereby created exciton may be disassociated at the P/N interface and the excited electron created during irradiation may thus be conveyed by the type N semi-conductor towards the anode, the hole being as for it led to the cathode via the type P semi-conductor.

Within the scope of the present description, the notion of a donor (type P semi-conductor) or acceptor (type N semi-conductor) nature of a semi-conducting compound is relative and depends on the nature of the compound with which it is associated within the photovoltaic coating. A compound including attractor groups is generally of the acceptor nature (type N), and conversely a compound including donor groups is generally of a donor nature (type P).

Photovoltaic devices applying organic semi-conductors are potentially promising. Indeed, considering the application of organic compounds of the polymer type as a replacement for inorganic semi-conductors, they provide the advantage of being more flexible mechanically and consequently less fragile, than the first and second generation systems. Moreover, they are more lightweight and they are further easier to make and prove to be less expensive.

However, to this day, the types of organic semi-conductor compounds used in photovoltaic devices are restricted, which is an obstacle to their actual use in the production of photovoltaic energy. Consequently, many efforts have been made for trying to diversify the types of organic semi-conducting compounds used.

Presently, organic semi-conductor compounds of type P (electron donor) used are generally of the polythiophene type, such as for example poly(3-hexylthiophene) so-called P3HT, or of the poly(arylene vinylene) type, while the organic semi-conductor compounds of type N (electron acceptor) used are generally of the fullerene type, such as for example phenyl-C₆₁-butyric acid methyl ester, so-called PCBM.

Mixtures of the P3HT/PCBM type have for example been described in patent applications US2008/315187 or US2009/032808. Semi-conducting compounds of type P of the poly(arylene vinylene) type have for example been described in patent application WO94/29883.

An object of the present invention is to increase the variety of available coatings with a photovoltaic nature and to provide a novel type of coating with a photovoltaic nature, so as to be able to adapt their composition to the encountered needs.

For this purpose, the present invention provides a coating with a photovoltaic nature based on a mixture of organic semi-conductor compounds; of the N type (acceptor) and of the P type (donor), giving the possibility by selecting the donor/acceptor pair, of modulating the semi-conducting properties of the photovoltaic coating in a way adapted to the use of the latter within the scope of a photovoltaic device.

More specifically, the object of the present invention is a photovoltaic coating based on a mixture of at least one organic semi-conducting compound of type N and of at least one organic semi-conducting compound of type P, wherein at least one of the organic semi-conducting compounds, preferably the organic semi-conducting compound of type N, is a compound comprising a quinone core, preferably a compound of formula (I):

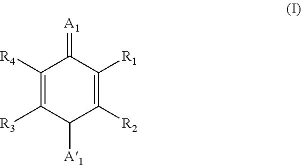

wherein:
the =A₁ group is a ═O, ═C(CN)₂ or ═N(CN) group;
each of the groups R₁, R₂, R₃ and R₄ independently represent a hydrogen atom, a halogen atom (F, Cl, Br), an amino, notably —NH₂ group, a —CN group, a —SO₂CF₃ group, an O-alkyl group, an O-aryl group, a hydrocarbon group (for example a linear or branched C₁-C₁₂ alkyl group, or an optionally substituted aryl group) or a polymeric chain which may contain several quinone groups;
it being understood that two or more of the groups R₁, R₂, R₃ and R₄ may form together an aromatic or heteroaromatic polycyclic structure;
the =A′₁ group is a ═O, ═C(CN)₂ or ═N(CN) group, generally identical with =A₁ or else a ={A″₁}=A₁ group wherein the {A″₁} group is an aromatic cyclic unit, it being understood that A′₁, R₂ and/or R₃ may form together an aromatic polycyclic structure.

According to a specific embodiment, the coating according to the invention is a coating with a photovoltaic nature based on a mixture of at least one organic semi-conducting compound of type N and of at least one organic semi-conducting compound of type P, wherein at least one of the organic semi-conducting compounds, preferably the organic semi-conducting compound of type N, fits formula (I).

Alternatively, the coating according to the invention may apply compounds comprising a quinone core other than the compounds of formula (I), for example compounds of the anthraquinone type, optionally substituted, or else compounds comprising a quinone or anthraquinone core and at least one substituent with a donor nature, such as a thiophene or a polythiophene.

In the coating of the invention, the organic semi-conducting compounds of type N and P are able to provide, in an association, a photovoltaic effect.

Preferably, the coating of the present invention is an exclusively organic coating, which generally contains the mixture of at least one organic semi-conducting compound of type N and of at least one organic semi-conducting compound of type P, notably excluding inorganic semi-conducting compounds. According to a particular embodiment, the coating of the present invention consists in a mixture comprising one or several organic semi-conducting compounds of type N, one or several organic semi-conducting compounds of type P, and optionally organic additives. According to a still more specific embodiment, the coating of the present invention is a coating exclusively consisting in a mixture of one or several organic semi-conducting compounds of type N, and one or several organic semi-conducting compounds of type P.

Moreover, regardless of its composition, the coating of the invention is preferably a coating comprising the organic semi-conducting compounds of type N and P in association within a same layer. Preferably, this is a mono-layer coating.

The coating according to the invention has the advantage of being able to adjust the energy of the LUMO of the semi-conducting compound of the acceptor type according to the energy of the HOMO of the semi-conducting compound of the donor type, and vice versa. Indeed, by the variety of semi-conducting organic compounds of formula (I), notably because of the variety of the R₁-R₄ groups which may have a donor or attractor nature, the invention makes available a large range of semi-conducting compounds, which may both play the role of a compound of the acceptor type or of the donor type, according to the nature of the R₁-R₄ groups, and of that of the compound with which they are associated within the coating according to the invention.

Generally, within the scope of the present description, the compound of formula (I) is a semi-conducting compound having an acceptor nature (of type N).

However, a compound of formula (I) may also play the role of a donor semi-conducting compound (of type P) when it is associated with a semi-conducting compound having a better acceptor nature.

According to the present invention, the <<alkyl>> radicals represent saturated hydrocarbon radicals, with a linear or branched chain, comprising from 1 to 10 carbon atoms, preferably from 1 to 5 carbon atoms (they may typically be represented by the formula CₙH₂ₙ₊₁, n representing the number of carbon atoms). Mention may notably be made, when they are linear, of the methyl, ethyl, propyl, butyl, pentyl, hexyl, octyl, nonyl and decyl radicals. Mention may notably be made, when they are branched or substituted with one or several alkyl radicals, of the isopropyl, tertbutyl, 2-ethylhexyl, 2-methylbutyl, 2-methylpentyl, 1-methylpentyl and 3-methylheptyl radicals.

The term of <<halogen>> designates a bromine, chlorine, fluorine or iodine atom.

The term of <<aryl>> designates a mono- or bi-cyclic hydrocarbon aromatic system comprising from 6 to 30, preferably from 6 to 10, carbon atoms. Among the aryl radicals, mention may notably be made of the phenyl or naphthyl radical, more particular substituted with at least one halogen atom or an —OH group. When the aryl radical comprises at least one heteroatom, this is referred to as a <<heteroaryl>> radical. Thus, the term of <<heteroaryl>> designates an aromatic system comprising one or several heteroatoms selected from nitrogen, oxygen or sulfur, either mono- or bi-cyclic, comprising from 5 to 30, and preferably from 5 to 10 carbon atoms. Among heteroaryl radicals, mention may be made of pyrazinyl, thienyl, oxazolyl, furazanyl, pyrrolyl, 1,2,4-thiadiazoyl, naphthyridininyl, pyridazinyl, quinoxalinyl, phthalazinyl, imidazo[1,2-a]pyridine, imidazo[2,1-b]thiazolyl, cinnolinyl, triazinyl, benzofurazanyl, azalindolyl, benzimidazolyl, benzothienyl, thienopyridyl, thienopyrimidinyl, pyrrolopyridyl, imidazopyridyl, benzoazaindole, 1,2,4-triazinyl, benzothiazolyl, furanyl, imidazolyl, indolyl, triazolyl, tetrazolyl, indolizinyl, isoxazolyl, isoquinolinyl, isothiazolyl, oxadiazolyl, pyrazinyl, pyridazinyl, pyrazolyl, pyridyl, pyrimidinyl, purinyl, quinazolinyl, quinolinyl, isoquinolyl, 1,3,4-thiadiazolyl, thiazolyl, triazinyl, isothiazolyl, carbazolyl, as well as the corresponding groups from their condensation or condensation with the phenyl group. The aforementioned <<alkyl>>, <<aryl>> and <<cycloalkyl>> radicals may be substituted with one or several substituents. Among the substituents, mention may be made of the following groups: amino, hydroxyl, thio, halogen, alkyl, alkoxy, alkylthio, alkylamino, aryloxy, arylalkoxy, cyano, trifluoromethyl, carboxy or carboxyalkyl.

The term of <<aromatic polycyclic structure>> designates a bi-, tri- or poly-cyclic structure consisting of aryl units placed side by side.

The term of <<heteroaromatic polycyclic structure>> designates a bi-, tri- or poly-cyclic structure consisting of heteroaryl units placed side by side.

According to a first particular embodiment of the invention, the organic semi-conducting compound of formula (I), which is preferably of the N type, is such that:
the groups =A′₁ and =A₁ are identical and represent a ═O, ═C(CN)₂ or ═N(CN) group; and
the groups R₁ and R₂ and optionally the groups R₃ and R₄ form together with the two carbon atoms to which they are bound, an aromatic or heteroaromatic, cyclic or polycyclic structure, optionally substituted, preferably a benzene, thiophene, thiadiazole, naphthalene, benzothiophene, benzothiazole, naphthothiophene, anthracene, or a combination of these structures.

The compounds of formula (I) according to this first embodiment may for example be selected from the following compounds:

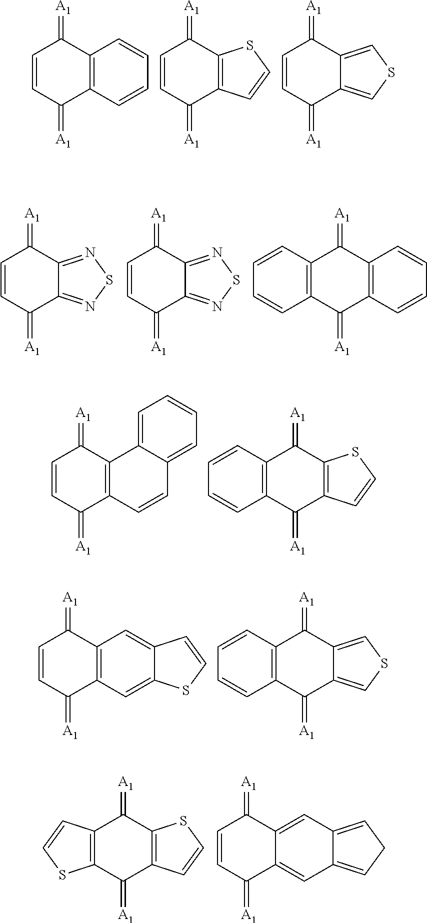

According to a second embodiment of the invention, the organic semi-conducting compound of formula (I), which is preferably of the N type, is such that:
the groups =A₁ and =A′₁ are identical and represent a ═O, ═C(CN)₂ or ═N(CN) group; and
R₁, R₂, R₃, R₄ are identical and represent an atom or a group selected from: H, F, Cl, —CN, —OMe, —OPh, —O—C₈H₄—OH, an unsaturated or saturated hydrocarbon chain.

The compounds of formula (I) according to this second embodiment may for example be selected from the following compounds:

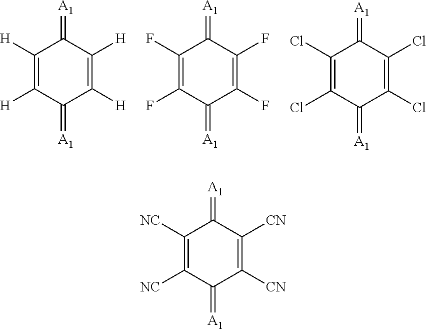

or for example selected from natural quinones such as ubiquinone:

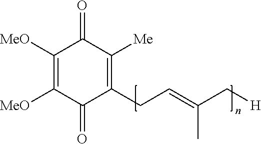

or for example from the following compounds which have a <<πstacking >> effect between the different aromatic rings:

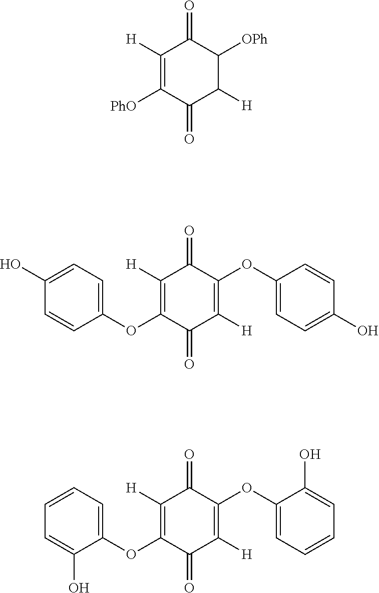 [SMILES: O=C1CC(Oc2ccccc2)C(=O)C=C1Oc1ccccc1 ; O=C1C=C(Oc2ccc(O)cc2)C(=O)C=C1Oc1ccc(O)cc1 ; O=C1C=C(Oc2ccccc2O)C(=O)C=C1Oc1ccccc1O]

According to a third embodiment of the invention, the organic semi-conducting compound of formula (I), which is preferably of the N type, is such that:
the group =A′₁ is a group ={A″₁}=A₁, wherein the group {A″₁} is an aromatic cyclic unit, it being understood that A′₁, R₂ and/or R₃ may form together an aromatic or heteroaromatic polycyclic structure, optionally substituted;
the groups R₁ and R₂ each represent H.

The compounds of formula (I) according to this third embodiment may for example be selected from the following compounds:

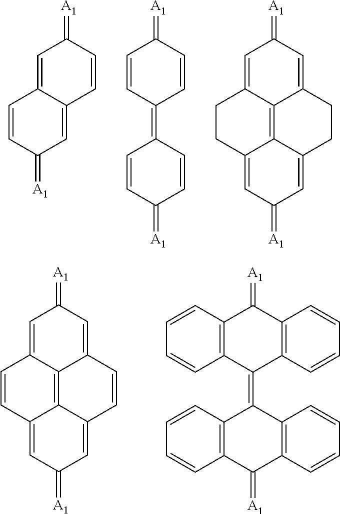

According to a fourth embodiment of the invention, R₁ and R₂ are polymeric chains such that the organic semi-conducting compound of formula (I), which is preferably of type N, is an oligomer comprising elementary units of formula (Ia):

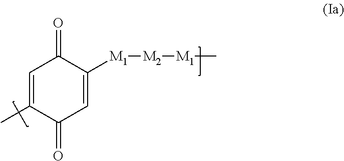

wherein:
the group M₁ represents a group —NH— or else a bond, and
the group M₂ represents a group —Ph-, —Ph-Ph-, —Ph-Ph-Ph-, or else a naphthalene group, each benzene ring Ph being optionally substituted with one or even two alkyl groups.

Such compounds are notably described in Dulov et. al. (Russian Chem. Rev (1966), 35. No. 10, 773).

The coating according to the invention comprises in association with the compound of formula (I), an organic semi-conductor of formula (II). This semi-conductor of formula (II) is of type N when the semi-conducting compound of formula (I) is of type P. This semi-conductor of formula (II) is of type P when the semi-conducting compound of formula (I) is of type N.

According to this last alternative, when the organic semi-conducting compound of formula (I) is of type N, the organic semi-conducting compound of formula (II) has a structure defined according to the following formula (II-1):

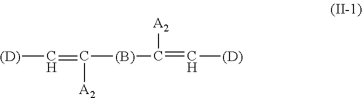

wherein:
A₂ represents a group selected from the group consisting of H, F, CN, CF₃, COORᵢ, CONRᵢRᵢᵢ wherein Rᵢ and Rᵢᵢ independently represent H or an alkyl group;
the group {B} represents an aromatic cyclic group, typically a phenylene, optionally substituted;
the group {D} represents a heteroaromatic or organo metallic, aromatic cyclic hydrocarbon group, optionally substituted, or else a polymeric chain.

The organic semi-conducting compounds of formula (II-1) are typically of type P.

According to an advantageous embodiment, the group {B} is a phenylene substituted with two O-alkyl groups, either identical or different, generally identical, for example OC₈H₁₃ or OC₁₂H₂₅, or a linear or branched alkyl, for example methyl or isopropyl.

Depending on the position of the O-alkyl groups, either in the para or ortho position, the group {B} respectively has a hydroquinone or pyrocatechin structure:

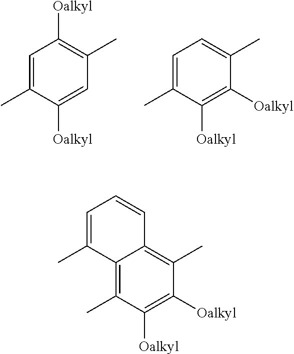

According to another alternative, the group {B} is a thiophene, optionally substituted with an alkyl group, for example C₁₂H₂₅.

The group {B} represents an aromatic cyclic group and preferably represents a monocyclic group, such as a phenyl or a thiophene, or else a polycyclic group, such as an anthracene or a carbazole.

The group {D} represents an organometallic or heteroaromatic, aromatic, cyclic hydrocarbon group, having aromaticity nature, and preferably represents a monocyclic group, such as a phenyl, a heterocyclic group, such as a thiophene, a furane, or a polycyclic group like an anthracene, an indole, a quinoline, a carbazole, a fluorine or a combination of these structures.

The group {D} may be selected from the following groups:

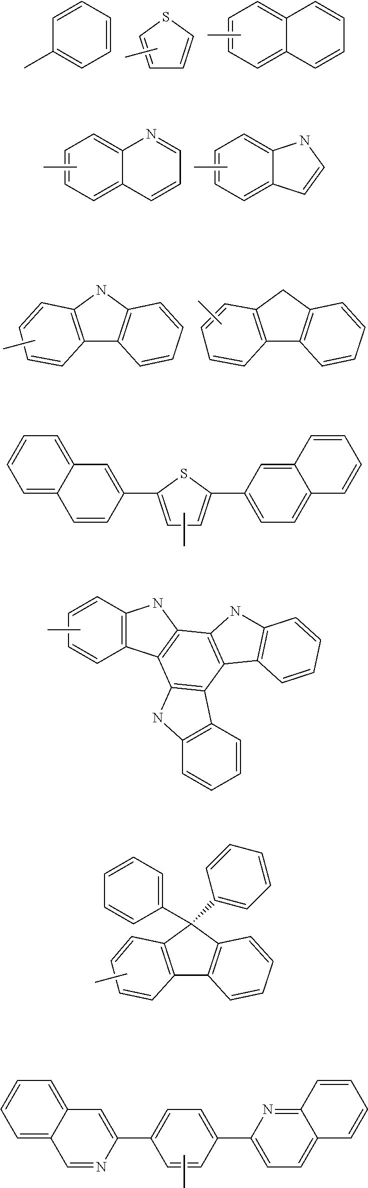

According to a particular embodiment, the group {D} represents an organometallic, heteroaromatic, or aromatic, cyclic hydrocarbon group optionally substituted, such as for example a phenyl group, optionally substituted with two O-alkyl groups, for example OC₆H₁₃ or OC₁₂H₂₅, or a thiophene group, optionally substituted with an alkyl group, for example C₁₂H₂₅, or a furane group, optionally substituted with an alkyl group, or further an indole, carbazole, ferrocenium or naphthalene group, optionally substituted with an alkyl group.

According to another alternative, the organic semi-conducting compound of formula (II) is an oligomer comprising elementary units of formula (II-2):

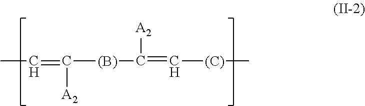

wherein:
A₂ and {B} are defined as earlier; and
the group {C} represents an aromatic or heteroaromatic, cyclic hydrocarbon group, optionally substituted.

The organic semi-conducting compounds of formula (II-2) are typically of type P.

According to an advantageous embodiment, the group {C} represents a phenylene group, optionally substituted with two —O-alkyl groups, either identical or different, generally identical, for example —OC₆H₁₃ or —OC₁₂H₂₅, or else a thiophene group, optionally substituted with an alkyl group, for example C₁₂H₂₅, or further a carbazole group optionally substituted with an alkyl group, for example C₁₂H₂₅.

According to another alternative, when the organic semi-conducting compound of formula (I) is type N, the organic semi-conducting compound of formula (II) is an oligomer comprising elementary units of formula (II-3):

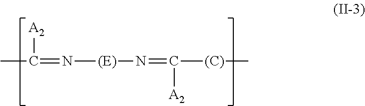

wherein:
A₂ and {C} are defined as earlier; and
the group {E} represents an aromatic or heteroaromatic ring, optionally substituted.

According to an advantageous embodiment, the group {E} is a thiophene substituted with two ester groups, either identical or different, generally identical, for example —COOR with R being a C₈-C₁₂ alkyl.

According to a particular embodiment, the semi-conducting compound of formula (II) is an oligomer fitting the formula (II-4):

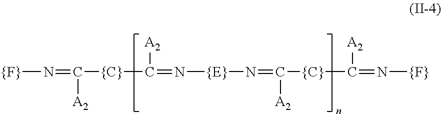

wherein:
the groups A₂, {C} and {E} are defined as earlier;
the group {F} represents an aromatic or heteroaromatic ring, optionally substituted; and
n is comprised between 1 and 15.

According to an advantageous embodiment, the group {F} is a phenylene, optionally substituted with an alkyl or —O-alkyl group.

According to another alternative, the coating according to the invention comprises an organic semi-conducting compound, typically of type N, fitting the formula (I′) and an organic semi-conducting compound, typically of type P, fitting the formula (III):

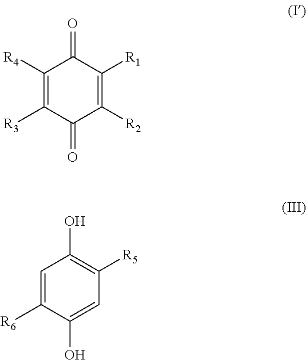

wherein:
each of the groups R₁, R₂, R₃ and R₄ independently represents a hydrogen atom, a halogen atom (F, Cl, Br), a —CN group or a hydrocarbon group (for example a linear or branched C₁-C₁₂ alkyl group, or an optionally substituted aryl group),
it being understood that two or more of the groups R₁, R₂, R₃ and R₄ may form together an aromatic or heteroaromatic polycyclic structure;
each of the groups R₅ and R₆ represent independently an H, hydroxyl, alkyl, —O-alkyl or aryl group,
it being understood that R₅ and/or R₆ may form with the central benzene ring an aromatic or heteroaromatic polycyclic structure.

According to an advantageous embodiment, the groups R₅ and R₆ are identical and represent H, a C₁-C₁₂ alkyl group, a C₁-C₁₂ O-alkyl group, or an aryl group, such as for example a phenyl.

According to another embodiment, the phenol functions of the compounds of formula (III) are in the form of arylether, for example as a compound of the following formula (III′):

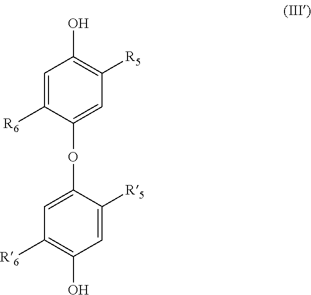

wherein: the groups R′₅ and R′₆ are defined in the same way as the groups R₅ and R₆.

According to a particular embodiment, the groups R₁, R₂, R₃ and R₄ are identical and represent H, F, Cl or CN, and the groups R₅ and R₆ are identical and represent a H, hydroxyl, typically C₁-C₈, alkyl or —O-alkyl group.

The compounds of formula (III) or (III′) according to this embodiment may be selected from the following compounds:

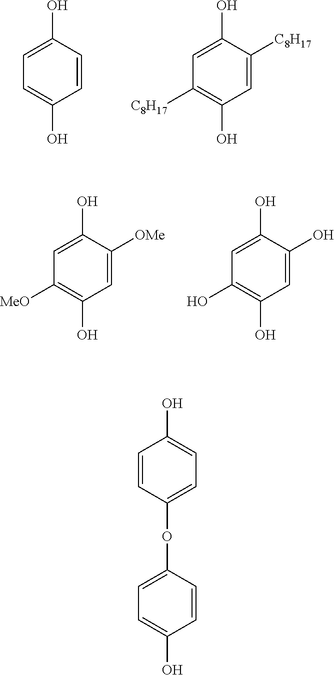

The compounds of formula (I′) according to this embodiment may be selected from the following compounds:

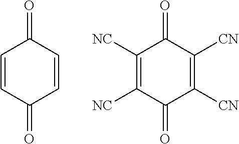 [SMILES: O=C1C=CC(=O)C=C1 ; N#CC1=C(C#N)C(=O)C(C#N)=C(C#N)C1=O]

According to another particular embodiment, three groups from among the groups R₁, R₂, R₃ and R₄ represent H, while the fourth group represents Me or Ph, and a group from among the groups R₅ and R₆ represent H, while the other one represents Me or Ph.

The compounds of formula (III) according to this embodiment may be selected from the following compounds:

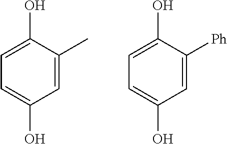

The compounds of formula (I′) according to this embodiment may be selected from the following compounds:

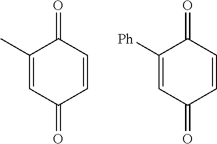

According to another particular embodiment, the groups R₁ and R₂ form together an aromatic or heteroaromatic polycyclic structure, and/or the group R₅ forms with the benzene ring an aromatic or heteroaromatic polycyclic structure.

The compounds of formula (III) according to this embodiment may be selected from the following compounds:

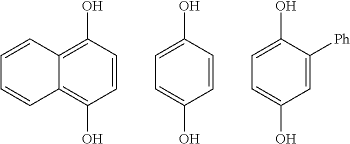

The compounds of formula (I′) according to this embodiment may be selected from the following compounds:

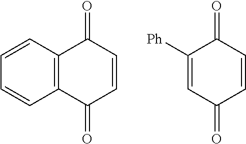

Preferably, the compounds of formula (I′) and (III) are associated within the coating, generally as quinhydrones.

For example the association of benzoquinone and of hydroquinone forms a quinhydrone, as illustrated hereafter:

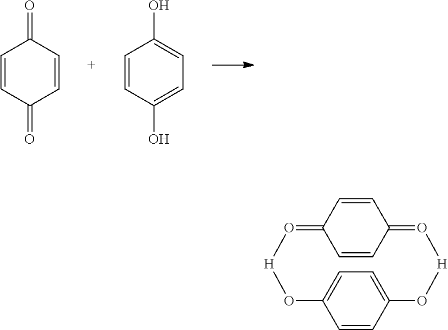

The invention also relates to the organic oligomeric compounds fitting the formula (IV):

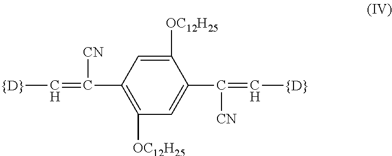

wherein:
the group {D} represents an organometal or heteroaromatic, aromatic cyclic hydrocarbon group, optionally substituted.

Within the scope of the present description, the compounds of formula (IV) are generally of type P, but may play the role of compound of type N under certain conditions, notably when they are associated with compounds having a better type P nature.

Typically, according to this aspect of the invention, the group {D} represents a thiophene, furane, anthracene or ferrocenium group, optionally substituted with an alkyl group.

The group {D} according to this embodiment may be for example selected from the following groups:

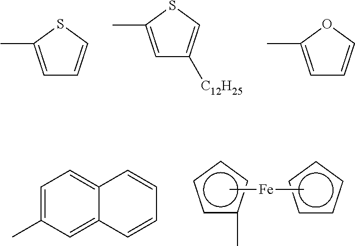

The invention also relates to the organic oligomeric compounds comprising elementary units of formula (V):

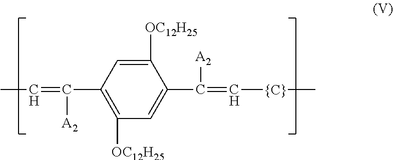

wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {C} represents a aromatic or heteroaromatic cyclic hydrocarbon group, optionally substituted, or a polymeric chain.

Within the scope of the present description, the organic oligomeric compounds comprising elementary units of formula (V) are generally of type P, but may play the role of compounds of type N under certain conditions, notably when they are associated with compounds having a better type P nature.

Typically, according to this aspect of the invention, the group {C} represents a phenyl, thiophene or carbazole group optionally substituted with an alkyl or O-alkyl group.

The group {C} according to this embodiment may be for example selected from the following groups:

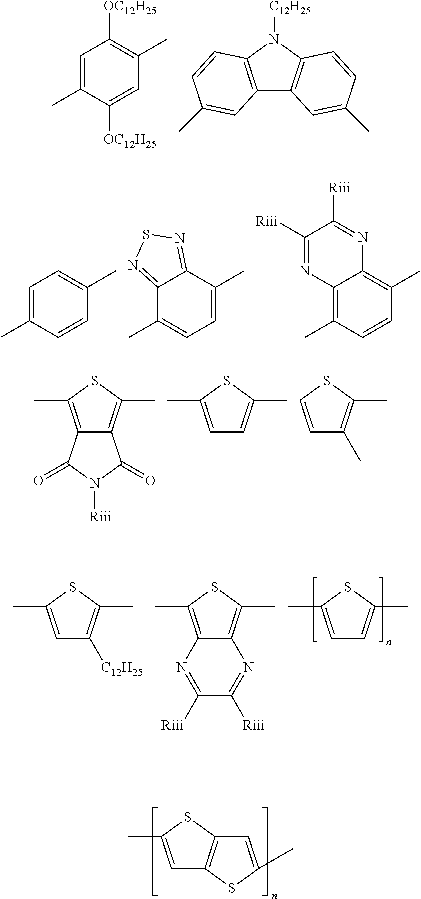

wherein n is comprised from 1 to 15, preferably equal to 1 or 2, and wherein Rᵢᵢᵢ represents H, an alkyl group, for example C₁₂H₂₅, or an aryl group for example —Ph, optionally substituted with an —OMe group.

### EXAMPLES

### Example 1

### Preparation of Photovoltaic Cells Comprising an Organic Photovoltaic Coating

Organic photovoltaic cells, including an organic layer with a semi-conducting nature, are prepared from acceptor and donor compounds according to the invention. More specifically, these cells are prepared under the conditions hereafter, notably described in patent application FR 0 956 641.

On a glass support (1 cm×1 cm plate) coated with a conducting layer of indium oxide doped with tin (ITO) (a commercial support provided with an ITO layer with a thickness of 100 nm), a layer of PEDOT:PSS (a charge collecting layer) with a thickness of 40 nm (obtained by spin coating and then by sol/gel texturation) was deposited

On the thereby prepared support, a photovoltaic coating was made from acceptor and donor compounds according to the invention.

To do this, 30 mg of the para-phenoxy-hydroquinone donor compound and 30 mg of the phenoxy-parabenzoquinone acceptor compound were dissolved in 3 ml of ortho-xylene so as to obtain a solution comprising 1% by mass of para-hydroxyphenoxy-hydroquinone and 1% by mass of phenoxy-parabenzoquinone in ortho-xylene. This solution was set under stirring at 70° C. for 24 hours in order to obtain complete solvation of the para-hydroxyphenoxy-hydroquinone and of the phenoxy-parabenzoquinone.

A mixture M comprising by mass 89% of dimethyl methylglutarate, 9% of dimethyl2-ethylsuccinate and 1% of dimethyl adipate obtained according to the method described below, was then added to the thereby obtained solution.

In a 500 ml glass reactor provided with an upward flowing refrigerant, with a stirrer and heated by an oil bath, were loaded 76.90 g of methanol and 43.26 g of a mixture consisting of 86.9% by weight of methylglutaronitrile, 11.2% by weight of ethylsuccinonitrile and 1.9% by weight of adiponitrile.

The reaction medium was then cooled to 1° C. and then 84.22 g of 98 weight % sulfuric acid were added. The reaction medium was then refluxed and maintained under these conditions for 3 hours.

Next, after cooling to 60° C., 63 g of water were added. The thereby obtained reaction medium was maintained at 65° C. for 2 hours.

117 g of additional water are then added, by which a biphasic reaction medium was obtained. After removing the excess methanol by evaporation, both phases were decanted. The recovered organic phase was washed once with an aqueous solution saturated with sodium chloride additived with ammonia in order to obtain a pH close to 7, and then a second time with an aqueous solution saturated with sodium chloride, and then distillation of the organic phase was carried out, by which a mixture M was obtained.

The solution comprising the phenoxy-parabenzoquinone/para-hydroxyphenoxy-hydroquinone mixture in the thereby obtained ortho-xylene mixture/M mixture, was deposited by spin coating, with a speed of rotation of the plate of 700 revolutions per minute for 1 minute at room temperature (25° C.).

After evaporating the solvents, a photovoltaic coating was obtained with a controlled structure, having a thickness of about 150 nm.

The ortho-xylene solvent/M mixture pair gives the possibility of obtaining during drying, second phase separation at a nanometric scale by differences in affinities towards the solvent medium of the pair (phenoxy-parabenzoquinone/para-hydroxyphenoxy-hydroquinone) as this is described in patent FR 0 956 641 which allows optimization of the photovoltaic performances of the device.

A thin layer of aluminum (thickness of the order of about 100 nm) was then deposited as a cathode on the coating thereby produced by the evaporation process. In order to finalize the nanostructuration of the active layer (phenoxy-parabenzoquinone/para-hydroxyphenoxy-hydroquinone), we proceed with heat treatment of the photovoltaic cell (150° C. for 10 mins) which thus allows an increase in the photovoltaic yield of the device.

The thereby prepared photovoltaic cell operates under usual illumination conditions.

Similar cells with good efficiency may be obtained under similar conditions by using the compounds of the following Examples 2 to 20 instead of the compounds used in Example 1.

A photovoltaic effect was also observed by using anthraquinones.

The following Examples describe the operating procedures for preparing compounds of formula (II-1), (II-2) and (II-3).

### Preparation of the Initial Compounds:

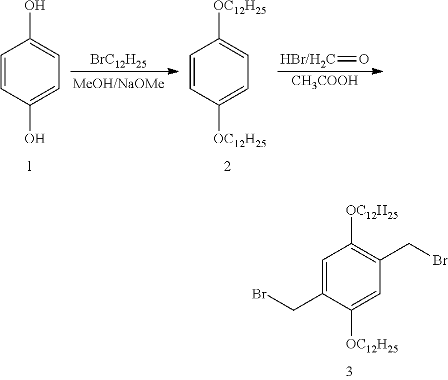

Under a nitrogen atmosphere, 120.0 g of hydroquinone (compound 1), 610.0 g of 1-bromodedecane and 1,000 mL of methanol are poured into a 3000 mL three-neck flask equipped with a thermometer, a condenser and a magnetic stirrer. 149.0 g of NaOMe are slowly added which causes an exothermic reaction (30-60° C.). The mixture is heated in an oil bath to about 60° C. for 16 hours and the reaction is tracked by thin film chromatography (TLC). When the initial compound has been totally consumed, the mixture is cooled to room temperature and then filtered. The filtered solid is washed with methanol and dried on a vacuum pump in order to obtain 415 g of a solid (compound 2, yield 85.2%).

Under a nitrogen atmosphere, 73.0 g of the compound 2, 160.0 mL of a 40% HBr solution in acetic acid and 580 mL of acetic acid are poured into a 1,000 mL three-neck flask equipped with a thermometer, with a condenser and with a magnetic stirrer. 14.72 g of paraformaldehyde are then added and the mixture is stirred at 70-75° C. for 2 hours. The mixture is then cooled to room temperature, filtered, washed with water with a pH of 7, washed with methanol and finally dried on a vacuum pump. The obtained solid is purified by recrystallization from 1,100 mL of n-hexane in order to obtain 93.0 g of a white solid (compound 3).

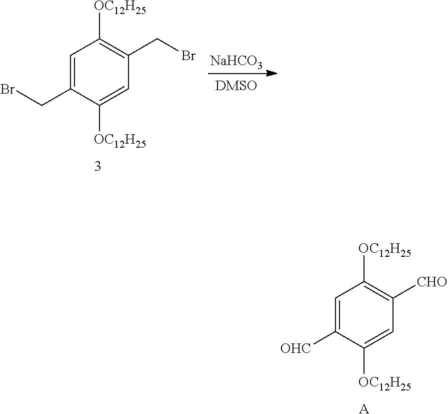

A mixture of compound 3 (95 g) and of sodium bicarbonate (190 g) in DMSO (2,400 mL) is stirred at 120° C. for 30 minutes. The reaction mixture is then poured into water (1,200 mL). The obtained precipitate is filtered and dried. The crude aldehyde mixture is purified by chromatography on silica gel by using a petroleum ether/dichloromethane mixture (20/1, V/V) as an eluent, which gives 9.0 g of a brown powder (compound 4) (which only reveals a single spot by TLC).

The structure of the compound is confirmed by ¹H NMR.

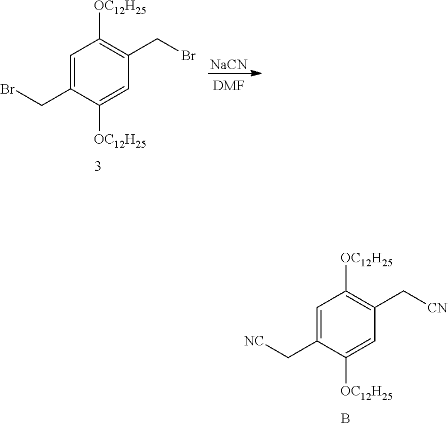

Under a nitrogen atmosphere, 79 g of compound 3, 1,080 mL of DMF and 15.6 g of NaCN are poured into a 2,000 mL three-neck flask equipped with a thermometer, a condenser and a magnetic stirrer. The mixture is stirred at 110-112° C. for 48 hours. The mixture is then cooled to room temperature, poured into 1,500 mL of a 0.5M NaOH solution and then filtered. The obtained crude mixture is dissolved in 1,500 mL of dichloromethane and washed with an aqueous solution saturated with NaCl (6 times 500 mL) in order to remove any trace amount of cyanide. The solvent is evaporated and the obtained black solid (68 g) is purified by recrystallization from 280 mL of a mixture of ethanol and chloroform (2/3, V/V) in order to obtain 15.8 g of a grey solid (compound 5).

The structure of the compound is confirmed by ¹HNMR.

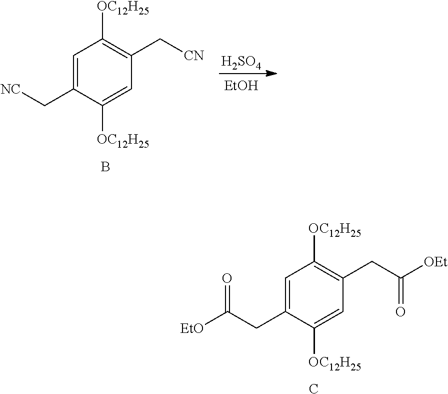

In a 250 mL three-necked flask, equipped with a thermometer, a condenser and a magnetic stirrer, are placed 40 mL of ethanol. The solvent is then cooled with a water/ice mixture, 14.8 g of concentrated sulfuric acid are added thereto, and then 20.0 g of the compound 5 so that the temperature of the mixture remains below 40° C. The mixture is stirred at 90° C. for 6 days. The mixture is then cooled, poured into a water/ice mixture, extracted with dichloromethane. The organic phase is dried and the solvents are evaporated in order to obtain 15.4 g of a crude solid. This crude solid is purified by chromatography on silica gel by using a hexane/dichloromethane mixture (1/1, V/V) as an eluent, in order to obtain 4.2 g of a white solid (compound 6).

The structure of the compound is confirmed by ¹HNMR.

### Preparation of Compounds of Formula (II-1) and (II-2):

### General Procedure:

For the preparation of an oligomeric compound of formula (II-1):

Under a nitrogen atmosphere, n millimoles of a compound for example including two —CH₂CN functions (typically the compound 5), n millimoles of a compound for example including two carbonyl functions, such as for example an aldehyde function or an ester function (typically the compound 4) and about 50 mL of tert-butanol are mixed.

The mixture is heated to 50° C. and t-BuOK is added. The mixture is left at this temperature until disappearance of the initial compounds. If necessary, extra additions of t-BuOK are performed.

The mixture is then cooled, and methanol is added. The precipitated solid is filtered and then washed with methanol and then possibly recrystallized.

For the preparation of a non-oligomeric compound of formula (II-1):

The same method is followed, by using an initial compound including a single carbonyl function.

### Example 2

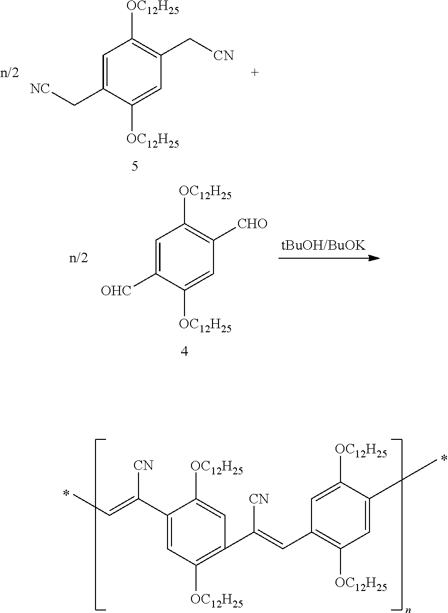

Under a nitrogen atmosphere, 1.88 g (3.6 mmol) of compound 5, 1.8 g (3.6 mmol) of compound 4 and 190 mL of tert-butanol are poured in a 250 mL three-neck flask equipped with a thermometer, a condenser and a magnetic stirrer. The mixture is heated to 50° C. and 0.19 g (1.7 mmol) of t-BuOK are added. The reaction mixture is stirred for 1 hour and cooled to room temperature. 1,000 mL of methanol are added and the mixture is filtered. The obtained filtered solid is washed with methanol and dried on a vacuum pump in order to obtain 2.6 g (yield 70.6%) of an orange solid (T_{melting}=104-111° C.).

The structure of the compound is confirmed by ¹HNMR.

### Example 3

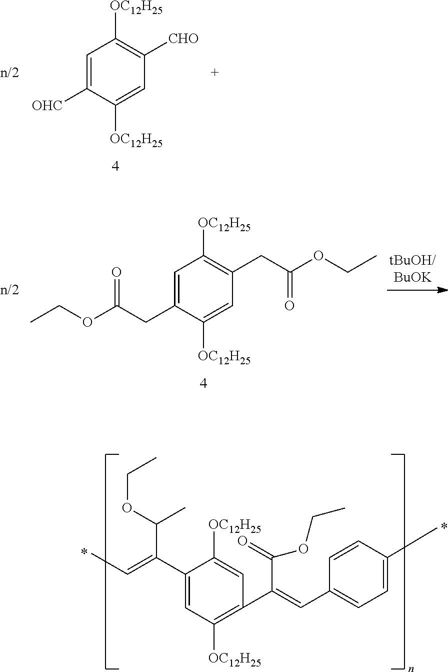

Under a nitrogen atmosphere, 2.5 g (4.97 mmol) of compound 5, 3.1 g (4.97 mmol) of compound 6 and 300 mL of tert-butanol are poured into a 500 mL three-neck flask equipped with a thermometer, a condenser and a magnetic stirrer. The mixture is heated to 50° C. and 0.3 g (2.7 mmol) of t-BuOK are added. The reaction mixture is stirred for 1 hour and cooled to room temperature. 1,500 mL of methanol are added and the mixture is filtered. The solvents are evaporated from the filtrate on a rotary evaporator in order to obtain 6.9 g of a red solid (T_{melting}=165-180° C., incomplete melting).

The structure of the compound is confirmed by ¹H and ¹³C NMR.

### Example 4

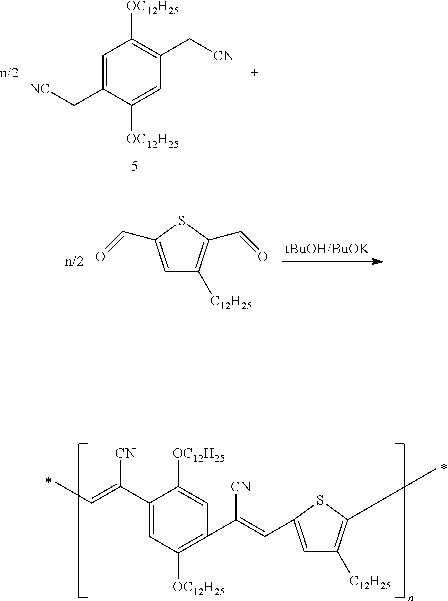

Under a nitrogen atmosphere, 4.0 g (7.6 mmol) of compound 5, 2.35 g (7.6 mmol) of 3-dodecyl-thiophene-2,5-dicarboxaldehyde and 400 mL of tert-butanol are poured into a 500 mL three-neck flask equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,000 mL of methanol are added and the mixture is filtered. The obtained filtered solid is dissolved in 80 mL of dichloromethane, and then 1,500 mL of ethyl acetate are added, which precipitates a solid. The obtained precipitated solid is filtered and dried on a vacuum pump in order to obtain 2.6 g of a brown solid (T_{melting}=170-200° C., incomplete melting).

The structure of the compound is confirmed by ¹³C NMR.

### Example 5

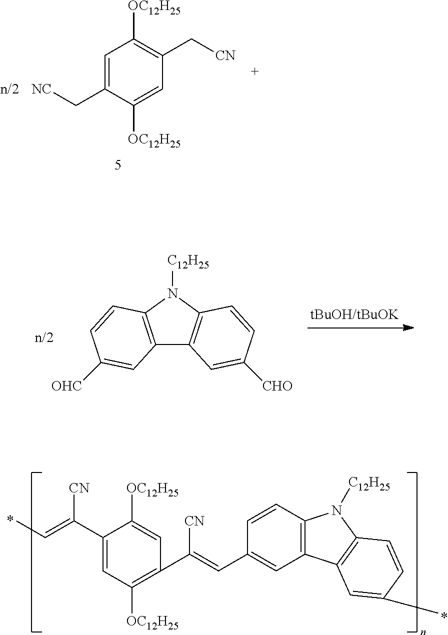

Under a nitrogen atmosphere, 4.0 g (7.6 mmol) of compound 5, 3.0 g (7.6 mmol) of 9-methyl-9H-carbazole-3,6-dicarboxaldehyde and 400 mL of tert-butanol are poured into a 500 three-neck flask equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,000 mL of methanol are added and the mixture is filtered. The obtained filtered solid is washed with methanol and dried on a vacuum pump in order to obtain 5.7 g of a crude solid. The obtained crude solid is dissolved in a 80 mL of dichloromethane, and then 1,000 mL of ethyl acetate are added, which precipitates a solid. The obtained precipitated solid is filtered and dried on a vacuum pump in order to obtain 3.2 g of a brown semi-solid.

The structure of the compound is confirmed by ¹H and ¹³C NMR.

### Example 6

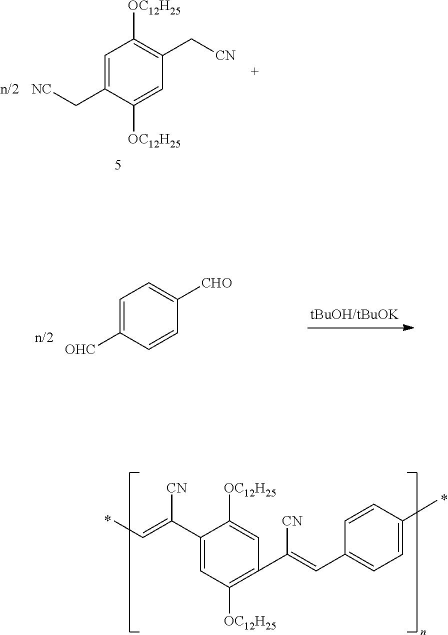

Under a nitrogen atmosphere, 4.0 g (7.6 mmol) of compound 5, 1.02 g (7.6 mmol) of para-benzyldialdehyde and 400 mL of tert-butanol are poured into a 500 mL three-neck flask equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,000 mL of methanol are added and the mixture is filtered. The obtained filtered solid is washed with methanol and dried on a vacuum pump in order to obtain 5.0 g of a crude solid. The obtained crude solid is dissolved in 150 mL of dichloromethane, and then 750 mL of ethyl acetate are added, which precipitates a solid. The obtained precipitated solid is filtered and dried on a vacuum pump in order to obtain 2.4 g of an orange solid (T_{melting}=232-300° C., incomplete melting).

The structure of the compound is confirmed by ¹H and ¹³C NMR.

### Example 7

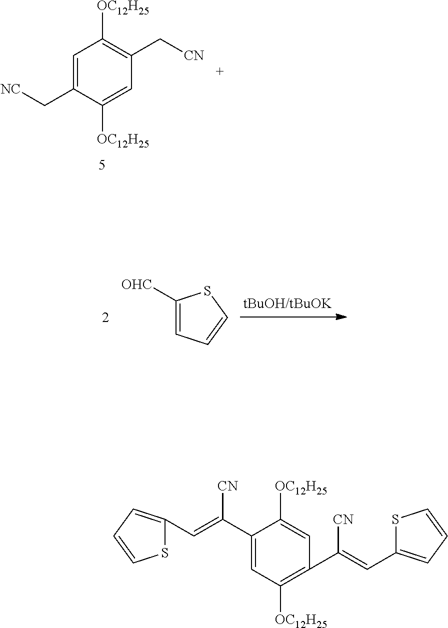

Under a nitrogen atmosphere, 10.0 g (19 mmol) of compound 5, 4.36 g (38 mmol) of thiophene-2-carbaldehyde and 400 mL of tert-butanol are poured into a 500 mL three-neck flask equipped with a thermometer, a condenser and a magnetic stirrer. The mixture is heated to 50° C. and 1.0 g (9.5 mmol) of t-BuOK are added. The reaction mixture is stirred for 1 hour and cooled to room temperature. 1,500 mL of methanol are added and the mixture is filtered. The obtained filtered solid is washed with methanol and dried on a vacuum pump in order to obtain 7.2 g of a crude solid. The solid is purified by recrystallization from 165 mL of ethyl acetate, and then washed with methanol and dried on a vacuum pump in order to obtain 5.8 g of a yellow solid.

The structure of the compound is confirmed by ¹H and ¹³C NMR.

### Example 8

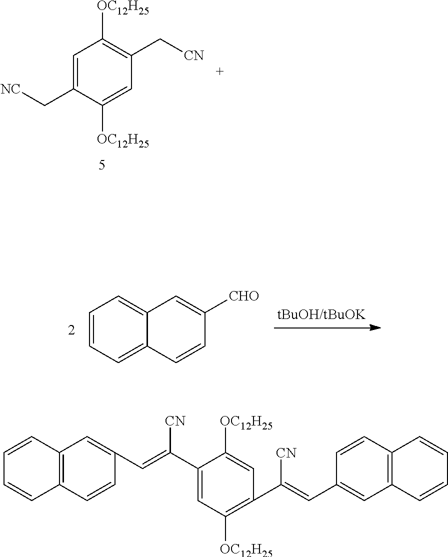

Under a nitrogen atmosphere, 4.0 g (19 mmol) of compound 5, 6.07 g (38 mmol) of 2-naphthaldehyde and 400 mL of tert-butanol are poured into a 500 mL three-neck flask equipped with a thermometer, a condenser and a magnetic stirrer. The mixture is heated to 50° C. and 1.0 g (9.5 mmol) of t BuOK are added. The reaction mixture is stirred for 1 hour and cooled to room temperature. 1,500 mL of methanol are added and the mixture is filtered. The obtained filtered solid is washed with methanol and dried on a vacuum pump in order to obtain 11.0 g of a crude solid. The solid is purified by crystallization from 165 mL of ethyl acetate and 35 mL of dichloromethane, and then washed with ethyl acetate and dried on a vacuum pump in order to obtain 9.5 g of a yellow solid (T_{melting}=179-190° C., incomplete melting).

The structure of the compound is confirmed by ¹H and ¹³C NMR.

### Example 9

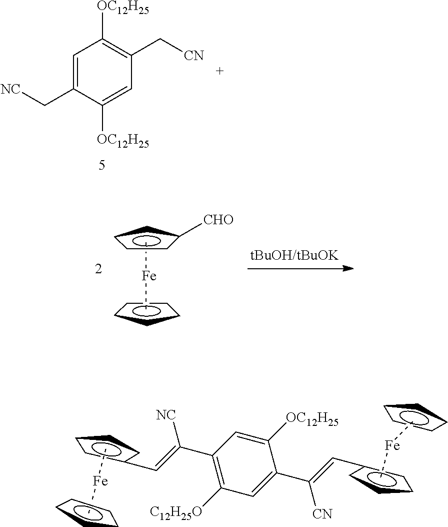

Under a nitrogen atmosphere, 4.0 g of compound 5, 3.26 g of ferrocene carboxaldehyde and 400 mL of tert-butanol in a 500 mL three-neck flask equipped with a thermometer, a condenser and a magnetic stirrer. The mixture is heated to 50° C. and 0.4 g of t-BuOK are added. The reaction mixture is stirred for 1 hour and cooled to room temperature. 1,500 mL of methanol are added and the mixture is filtered. The obtained filtered solid is washed with methanol and dried on a vacuum pump in order to obtain 4.0 g of a yellow solid.

The structure of the compound is confirmed by ¹H and ¹³C NMR.

### Example 10

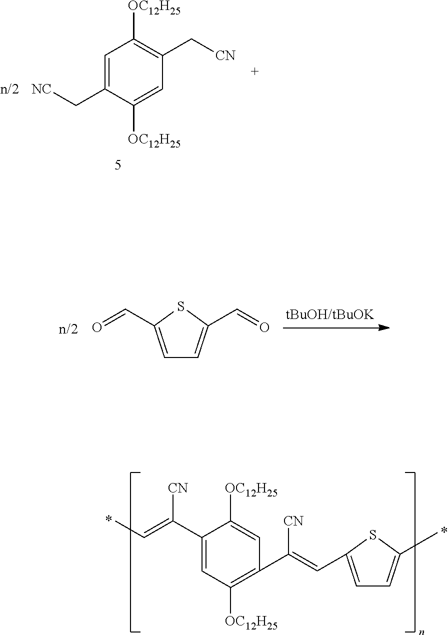

Under a nitrogen atmosphere, 4.0 g (7.6 mmol) of compound 5, 1.08 g (7.6 mmol) of 2,5-thiophenedicarboxaldehyde and 400 mL of tert-butanol are poured in a 500 mL three-neck flask equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,500 mL of methanol are added and the mixture is filtered. The obtained filtered solid is washed with methanol and dried on a vacuum pump in order to obtain 3.6 g of a brown solid. The obtained crude solid is dissolved in 150 mL of dichloromethane, and then 1,000 mL of ethyl acetate are added. The mixture is left at room temperature for 12 hours and then the obtained precipitated solid is filtered, washed with methanol and dried in vacuo in order to obtain 2.6 g of a brown solid (T_{melting}=180-200° C., incomplete melting).

The structure of the compound is confirmed by ¹H and ¹³C NMR.

### Example 11

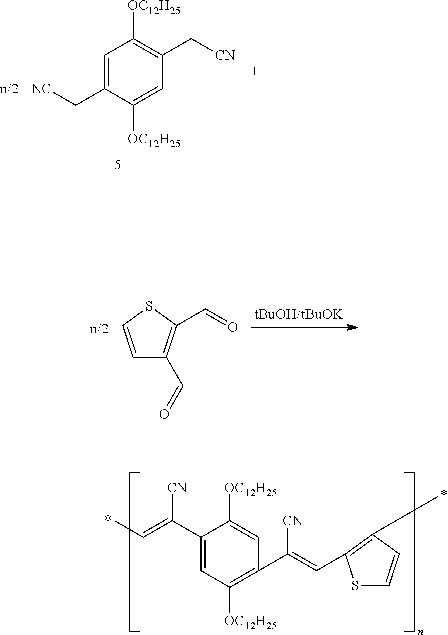

Under a nitrogen atmosphere, 4.0 g (7.6 mmol) of compound 5, 1.1 g (7.6 mmol) of 2,3-thiophenedicarboxaldehyde and 400 mL of tert-butanol are poured into a 500 mL three-neck flask equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,500 mL of methanol are added and the mixture is filtered. The obtained filtered solid is washed with methanol and dried on a vacuum pump in order to obtain 3.0 g of an orange solid. The obtained crude solid is dissolved in 100 mL of dichloromethane, and then 1,000 mL of ethyl acetate are added. The mixture is left at room temperature for 12 hours, and then the obtained precipitated solid is filtered, washed with methanol and dried in vacuo in order to obtain 2.2 g of a brown solid (T_{melting}=179-190° C., incomplete melting).

The structure of the compound is confirmed by ¹H and ¹³C NMR.

### Example 12

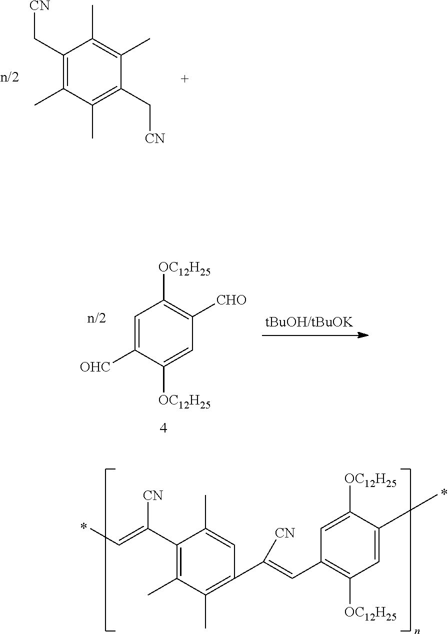

Under a nitrogen atmosphere, 2.0 g of (4-cyano-methyl-2,3,5,6-tetra-methyl-phenyl)-acetonitrile, 4.7 g of compound 4 and 500 mL of tert-butanol are poured into a 500 mL three-neck flask equipped with a thermometer, a condenser and a magnetic stirrer. The mixture is heated to 50° C. and 0.4 g of t-BuOK are added. The reaction mixture is stirred for 2 hours and cooled to room temperature. 1,500 mL of methanol are added and the mixture is filtered. The obtained filtered solid is washed with methanol and dried on a vacuum pump in order to obtain 3.0 g of a yellow solid. This solid is dissolved in 100 mL of dichloromethane and 100 mL of ethyl acetate, and then 1,000 mL of methanol are added. The mixture is left at room temperature for 12 hours, and then the obtained precipitated solid is filtered, washed with methanol and dried in vacuo in order to obtain 1.9 g of a yellow solid (T_{melting}=78-100° C., incomplete melting).

The structure of the compound is confirmed by ¹H and ¹³C NMR.

### Example 13

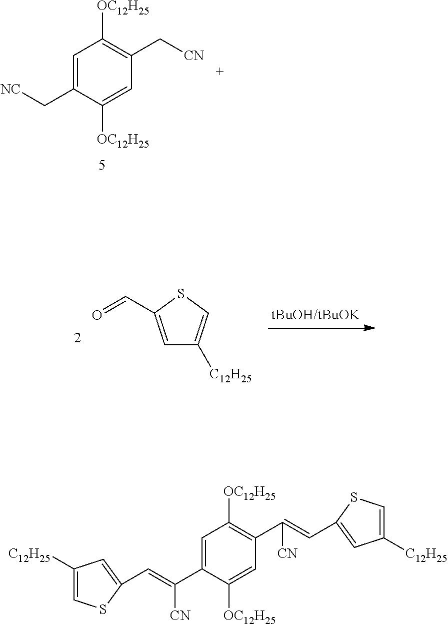

Under a nitrogen atmosphere, 4.0 g (7.6 mmol) of compound 5, 4.28 g (15.2 mmol) of 1-carboxaldehyde-3-dodecyl-thiophene and 400 mL of tert-butanol are poured in a 500 mL three-neck flask, equipped with a thermometer, a condenser and a magnetic stirrer. The mixture is heated to 50° C. and 0.4 g (3.6 mmol) of t-BuOK are added. The reaction mixture is stirred for 1 hour and cooled to room temperature. 1,500 mL of methanol and are added and the mixture is filtered. The obtained filtered solid is washed with methanol and dried on a vacuum pump in order to 5.0 g of a yellow solid. The obtained crude solid is dissolved in 200 mL of dichloromethane, and then 1,800 mL of ethyl acetate are added. The mixture is left at room temperature for 12 hours, and then the obtained precipitated solid is filtered, washed with methanol and dried in vacuo in order to obtain 4.2 g of a brown solid (T_{melting}=107-170° C., incomplete melting).

The structure of the compound is confirmed by ¹H and ¹³C NMR.

### Example 14

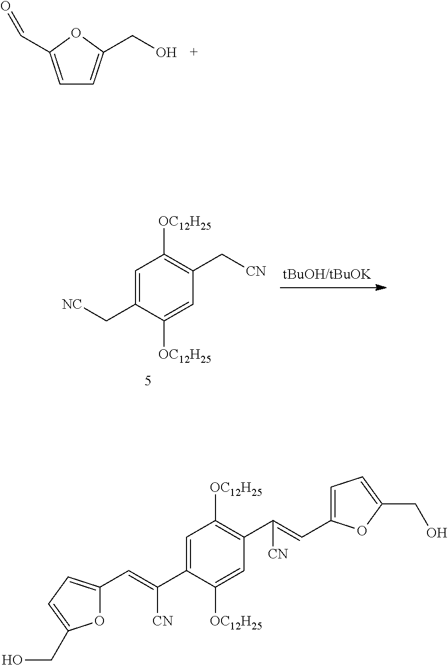

Under a nitrogen atmosphere, 10 g of compound 5, 6 g of 5-hydroxymethyl-furane-2-carbaldehyde and 800 mL of tert-butanol are poured into a 1,000 mL three-neck flask equipped with a thermometer, a condenser and a magnetic stirrer. The mixture is heated to 50° C. and 1.21 g of t-BuOK are added. The reaction mixture is stirred for 2 hours and cooled to room temperature. The solvent is evaporated under reduced pressure and a brown solid is obtained (16.6 g). The obtained solid is purified by chromatography on silica gel (eluent: petroleum ether/ethyl acetate 3:1) and an orange solid is obtained (8.4 g).

### Example 15

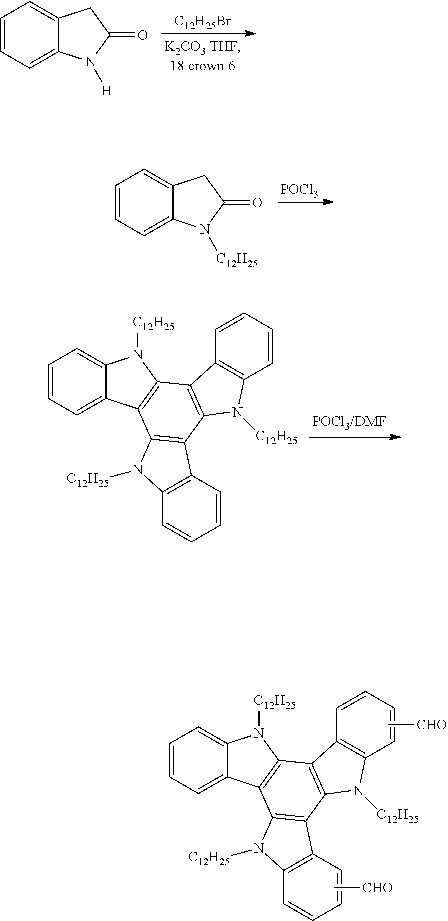

Step 1: A mixture of oxindole (50.0 g), of 1-bromododecane (186 g) of potassium carbonate (301.4 g) and of 18-crown-6-crown ether in 1,000 mL of THF is stirred under a nitrogen atmosphere at 30° C. The reaction is tracked by LC/MS and is stopped after 120 hours when the oxindole has completely reacted. The mixture is filtered, the solvents are evaporated and a red-brown solid is obtained (201 g). The crude mixture is purified by chromatography on silica gel by using a petroleum ether/dichloromethane mixture (20/1, V/V) as an eluent, in order to obtain 83 g of a brown solid.

Step 2: Under an air atmosphere, the obtained compound (181 g, 0.6 mol, 1 equivalent) is stirred and heated to 100° C. in 1,000 mL of POCl₃ (1,650 g, 17.9 equivalents). After consumption of the initial compound, POCl₃ is recovered under reduced pressure. Water and a NaOH solution are then added to the residue while cooling the mixture. The obtained crystals are filtered and dissolved in 1,500 mL of dichloromethane. The obtained solution is hot-filtered and the filtrate is washed with water, and then dried with MgSO₄ (289 g) overnight. The solvents are evaporated and the residue is purified by chromatography on silica gel by using a petroleum ether/ethyl acetate mixture (40/1 and then 20/1, V/V) as an eluent, in order to obtain 48 g of a brown oil.

Step 3: A solution of 48.0 g of the obtained compound in 600 mL of dichloroethane is slowly added, under a nitrogen atmosphere, to a mixture at 0° C. of 250 mL of DMF and 100 mL of dry POCl₃. The mixture is thoroughly stirred at 70° C. for 19 hours, and the reaction is then stopped by adding water. The mixture is extracted with chloroform (500 mL), filtered and dried on MgSO₄ (500 g). The solvents are evaporated on a rotary evaporator in order to obtain a black oil (70 g) and the residue is purified by chromatography on silica gel by using a petroleum ether/ethyl acetate mixture (40/1 and then 20/1, V/V) as an eluent.

The structure of the compound is confirmed by ¹H NMR.

### Example 16

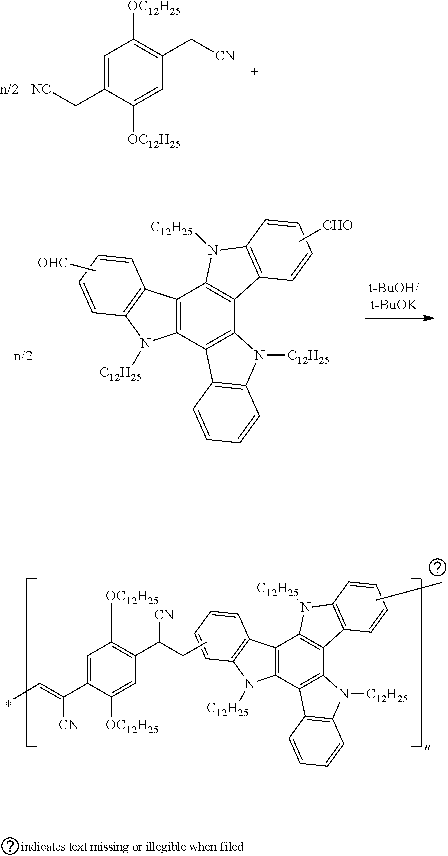

Under a nitrogen atmosphere, 3.0 g (5.7 mmol) of compound 5, 5.18 g (5.7 mmol) of compound prepared in Example 15 and 300 mL of tert-butanol are poured into a 500 mL three-neck flask equipped with a thermometer, a condenser and a magnetic stirrer. The mixture is heated to 50° C. and 0.3 g (2.7 mmol) of t-BuOK are added. The reaction mixture is stirred at this temperature for 16 hours, and then 0.3 g (2.7 mmol) of t-BuOK are again added. The reaction mixture is stirred for 20 hours at 70° C., and then 0.3 g (2.7 mmol) of t-BuOK are again added. The reaction mixture is stirred for 40 hours at 70° C. After disappearance of the compound B, the mixture is cooled to room temperature. 1,500 mL of methanol are added and the mixture is filtered. The obtained filtered solid is washed with 100 mL of methanol and dried on a vacuum pump in order to obtain 3.26 g of an orange solid.

The structure of the compound is confirmed by ¹H NMR.

### Preparation of Compounds of Formula (II-3):

### Example 17

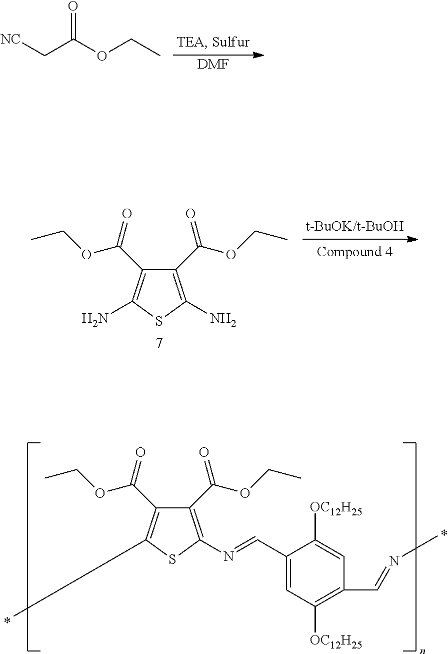

Step 1: In a 100 mL three-neck flask under a nitrogen atmosphere, equipped with a thermometer, a condenser and a magnetic stirrer, are placed 49.0 g of ethyl cyanoacetate, 35 mL of distilled DMF and 7 mL of distilled triethylamine. 7.0 g of sulfur are then added and the mixture is stirred at room temperature for 80 hours. The mixture is filtered and the filtrate is then poured into 1,500 mL of water. The solid form is filtered and dissolved in 2,000 mL of dichloromethane, dried on Na₂SO₄, the solvents are evaporated and a yellow solid (24.0 g) is obtained. This solid is stirred for 2 hours in 500 mL of n-hexane, the mixture is filtered and a yellow solid (22.2 g, 21.2%) is obtained (compound 7).

The structure of the compound is confirmed by ¹H NMR.

Step 2: Under a nitrogen atmosphere, 2.5 g of compound 7, 5.0 g of compound 4 and 500 mL of tert-butanol are poured into a 1,000 mL three-neck flask equipped with a thermometer, a condenser and a magnetic stirrer. The mixture is heated to 50° C. and 0.5 g of t-BuOK are added. The reaction mixture is stirred for 2 hours, and then 0.5 g of t-BuOK are again added. The reaction mixture is stirred for 16 hours, and then cooled to room temperature.

The solvents are evaporated under reduced pressure and an oily brown solid (8.8 g) is obtained. This crude solid is purified by chromatography on silica gel and an oily orange solid (0.8 g) is obtained.

The structure of the compound is confirmed by ¹H NMR.

### Example 18

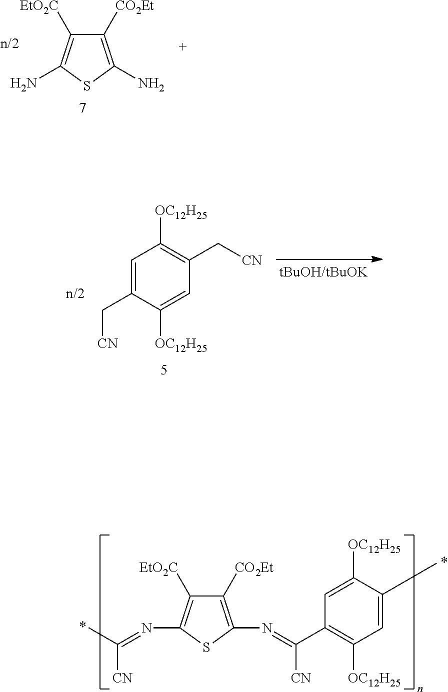

Under a nitrogen atmosphere, 18 g of compound 7, 9 g of compound 5 and 1,800 mL of tert-butanol are poured into a 2,000 mL three-neck flask equipped with a thermometer, a condenser and a magnetic stirrer. The mixture is heated to 50° C. and 3.6 g of t-BuOK are added. The reaction mixture is stirred for 18 hours and cooled to room temperature. The solvents are evaporated under reduced pressure and a brown solid is obtained (39 g). The obtained solid is dissolved in 1,600 mL of a mixture of petroleum ether and of ethyl acetate (15:1), the mixture is filtered, the filtrate is concentrated under reduced pressure down to a volume of about 500 mL, and then 1,400 mL of methanol are added and the mixture is concentrated down to a volume of about 600 mL. The mixture is cooled and the oil formed is separated from the mixture and purified by chromatography on silica gel and an orange solid is obtained (8.4 g). This solid is stirred for 2 hours in 200 mL of hexane, the mixture is filtered and an orange solid is obtained (4.8 g).

This solid is purified by chromatography on silica gel and an orange solid is obtained.

### Example 19

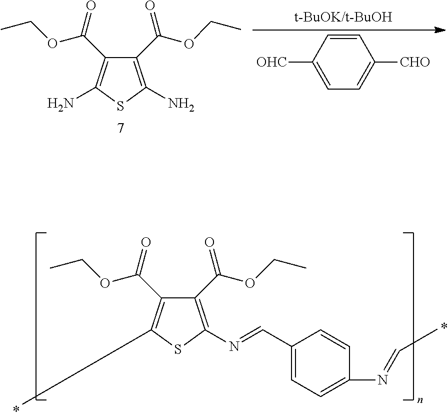

Under a nitrogen atmosphere, 20 g of compound 7, 10.4 g of terephthalaldehyde and 1,500 mL of tert-butanol are poured into a 2,000 mL three-neck flask equipped with a thermometer, a condenser and a magnetic stirrer. The mixture is heated to 50° C. and 4.0 g of t-BuOK are added. The reaction mixture is stirred at this temperature until one of the reagents (monitored by TLC) has disappeared. The solvents are evaporated under reduced pressure. To the obtained solid are added 50 mL of dichloromethane and 400 mL of methanol. The mixture is filtered, the obtained solid is washed with methanol in order to afford a yellow solid (3.1 g).

The structure of the compound is confirmed by ¹H NMR and by IR spectroscopy.

### Example 20

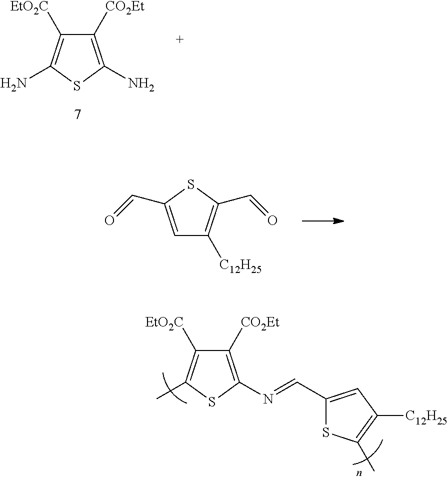

Under a nitrogen atmosphere, 1.76 g of compound 7, 2.1 g of 3-dodecyl-thiophene-2,5-dicarboxaldehyde and 130 mL of tert-butanol are poured into a 250 mL three-neck flask equipped with a thermometer, a condenser and a magnetic stirrer. The mixture is heated to 50° C. and 0.36 g of t-BuOK are added. The reaction mixture is stirred at this temperature until one of the reagents (monitored by TLC) has disappeared. The solvents are evaporated under reduced pressure. To the obtained solid, are added 20 mL of dichloromethane and 150 mL of methanol. The mixture is filtered, the obtained solid is washed with methanol in order to afford a brown semi-solid (3.5 g).

The structure of the compound is confirmed by ¹H NMR and by IR spectroscopy.

1. A photovoltaic coating, preferably exclusively organic, based on a mixture of at least one organic semi-conducting compound of type N and of at least one organic semi-conducting compound of type P, said coating preferably being a coating comprising said organic semi-conducting compound of type N and P in association within a same layer, and wherein at least one of the organic semi-conducting compounds, preferably the organic semi-conducting compound of type N, is a compound comprising a quinone core, preferably a compound of formula (I): 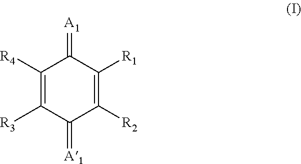
wherein:
the =A₁ group is a ═O, ═C(CN)₂ or ═N(CN) group;
each of the groups R₁, R₂, R₃ and R₄ represents independently a hydrogen atom, a halogen atom (F, Cl, Br), an amino group, notably —NH₂, a —CN group, a —SO₂CF₃ group, an O-alkyl group, a O-aryl group, or a hydrocarbon group (for example a linear or branched C₁-C₁₂ alkyl, or an optionally substituted aryl group);
it being understood that two or more of the groups R₁, R₂, R₃ and R₄ may form together an aromatic or heteroaromatic polycyclic structure;
the =A′₁ group is a ═O, ═C(CN)₂ or ═N(CN) group, generally identical with =A₁, or else a group ={A″₁}=A₁ wherein the group {A″₁} is an aromatic cyclic unit, it being understood that A′₁, R₂ and/or R₃ may form together an aromatic polycyclic structure.

2. The coating according to claim 1, wherein:
the group =A′₁ and =A₁ are identical and represent a group ═O, ═C(CN)₂ or ═N(CN); and
the groups R₁ and R₂ and optionally the groups R₃ and R₄ form together with the two carbon atoms to which they are bound, an aromatic or heteroaromatic, cyclic or polycyclic structure, optionally substituted, preferably a benzene, thiophene, thiadiazole, naphthalene, benzothiophene, benzothiazole, naphthothiophene, anthracene, or a combination of these structures.

3. The coating according to claim 1, wherein:
the =A₁ and =A′₁ groups are identical and represent a ═O, ═C(CN)₂ or ═N(CN) group; and
R₁, R₂, R₃ and R₄ are identical and represent an atom or a group selected from: H, F, Cl, CN, —OMe, —OPh, —O—C₆H₄—OH, a saturated or unsaturated hydrocarbon chain.

4. The coating according to claim 1, wherein:
the group =A′₁ is a group ={A″₁}=A₁ wherein the group {A″₁} is an aromatic cyclic unit, it being understood that A′₁, R₂ and/or R₃ may form together an aromatic polycyclic structure, optionally substituted;
the groups R₁ and R₂ each represent H.

5. The coating according to any of claim 1, also comprising an organic semi-conducting compound of formula (II-1): 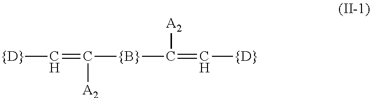
wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {B} represents an aromatic cyclic group, typically a phenylene, optionally substituted;
the group {D} represents an organometallic or heteroaromatic, aromatic cyclic hydrocarbon group, optionally substituted, or else a polymeric chain.

6. The coating according to claim 5, wherein the group {D} represents an organometallic, heteroaromatic, or aromatic, cyclic hydrocarbon group, optionally substituted, such as for example a phenylene group, optionally substituted with two O-alkyl groups, for example OC₆H₁₃ or OC₁₂H₂₅, or a thiophene group, optionally substituted with an alkyl group, or a furane group, optionally substituted with an alkyl group, for example C₁₂H₂₅, or further an indole, carbazole, ferrocenium or naphthalene group, optionally substituted with an alkyl group.

7. The coating according to claim 6, wherein the organic semi-conducting compound of formula (II-1) is an oligomer comprising elementary units of formula (II-2): 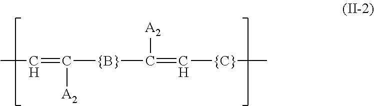
wherein:
A₂ represents a group selected from the group consisting in H, F, CN, CF₃, COOR and {B} represents an aromatic cyclic group, typically a phenylene, optionally substituted; and
the group {C} represents an aromatic or heteroaromatic cyclic hydrocarbon group, optionally substituted.

8. The coating according to any of claim 1, also comprising an oligomeric organic semi-conducting compound comprising elementary units of formula (II-3): 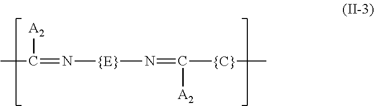
wherein:
the group A₂ represents a group selected from the group consisting in H, F, CN, CF₃, COOR and group {C} represents an aromatic or heteroaromatic cyclic hydrocarbon group, optionally substituted; and
the group {E} represents an aromatic or heteroaromatic ring, optionally substituted.

9. The coating according to claim 8, wherein the semi-conducting organic compound comprising elementary units of formula (II-3) is an oligomer fitting the formula (II-4): 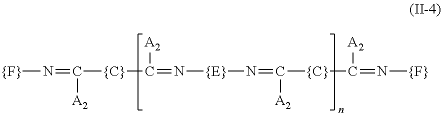
wherein:
the group A₂ represents a group selected from the group consisting in H, F, CN, CF₃, COOR, group {C} represents an aromatic or heteroaromatic cyclic hydrocarbon group, optionally substituted and group {E} represents an aromatic or heteroaromatic ring, optionally substituted;
the group {F} represents an aromatic or heteroaromatic ring, optionally substituted; and
n is comprised between 1 and 15.

10. The coating according to claim 1, comprising an organic semi-conducting compound, typically of type N, fitting the formula (I′) and an organic semi-conducting compound typically of type P, fitting the formula (III): 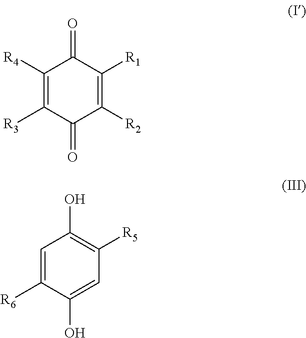
wherein:
each of the groups R₁, R₂, R₃ and R₄ represents independently a hydrogen atom, a halogen atom (F, Cl, Br), a —CN group, or a hydrocarbon group (for example a linear or branched C₁-C₁₂ alkyl group, or an aryl group optionally substituted),
it being understood that two or more of the groups R₁, R₂, R₃ and R₄ may form together an aromatic or heteroaromatic polycyclic structure;
each of the groups R₅ and R₆ represents independently a group H, hydroxyl, alkyl, O-alkyl or aryl,
it being understood that R₅ and/or R₆ may form with the central benzene ring, an aromatic or heteroaromatic structure.

11. The coating according to claim 10, wherein:
the groups R₁, R₂, R₃ and R₄ are identical and represent H, F, Cl or CN;
the groups R₅ and R₆ are identical and represent a group H or alkyl, typically a C₁-C₈ group.

12. The coating according to claim 10, wherein:
three groups from among the groups R₁, R₂, R₃ and R₄ represent H, while the fourth group represents Me or Ph;
a group from the groups R₅ and R₆ represents H, while the other one represents Me or Ph.

13. The coating according to claim 10, wherein:
the groups R₁, and R₂ form together an aromatic or heteroaromatic polycyclic structure, and/or
the group R₅ represents with the benzene ring an aromatic or heteroaromatic polycyclic structure.

14. An organic compound for making a coating according to claim 6, typically a semi-conductor of type P, fitting the formula (IV): 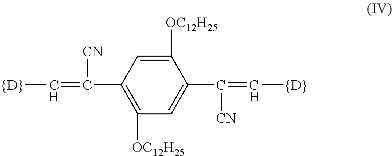
wherein the group {D} represents an organometallic or heteroaromatic, aromatic, cyclic hydrocarbon group, optionally substituted.

15. An organic oligomeric compound, for making a coating according to claim 7, typically a semi-conductor of type P, comprising elementary units of formula (V): 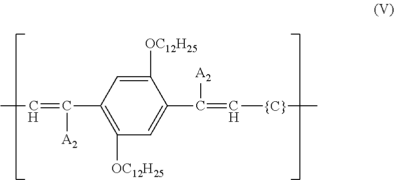
wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {C} represents an aromatic or heteroaromatic, cyclic hydrocarbon group, optionally substituted, or a polymeric chain.

16. The coating according to any of claim 2, also comprising an organic semi-conducting compound of formula (II-1): 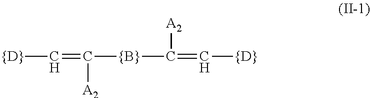
wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {B} represents an aromatic cyclic group, typically a phenylene, optionally substituted;
the group {D} represents an organometallic or heteroaromatic, aromatic cyclic hydrocarbon group, optionally substituted, or else a polymeric chain.

17. The coating according to any of claim 3, also comprising an organic semi-conducting compound of formula (II-1): 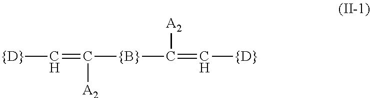
wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {B} represents an aromatic cyclic group, typically a phenylene, optionally substituted;
the group {D} represents an organometallic or heteroaromatic, aromatic cyclic hydrocarbon group, optionally substituted, or else a polymeric chain.

18. The coating according to any of claim 4, also comprising an organic semi-conducting compound of formula (II-1): 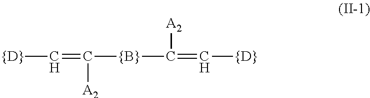
wherein:
A₂ represents a group selected from the group consisting in H, F, CN, CF₃, COORᵢ, CONRᵢRᵢᵢ wherein Rᵢ and Rᵢᵢ represent independently H or an alkyl group;
the group {B} represents an aromatic cyclic group, typically a phenylene, optionally substituted;
the group {D} represents an organometallic or heteroaromatic, aromatic cyclic hydrocarbon group, optionally substituted, or else a polymeric chain.

19. The coating according to any of claim 2, also comprising an oligomeric organic semi-conducting compound comprising elementary units of formula (II-3): 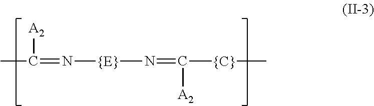
wherein:
the group A₂ represents a group selected from the group consisting in H, F, CN, CF₃, COOR and group {C} represents an aromatic or heteroaromatic cyclic hydrocarbon group, optionally substituted; and
the group {E} represents an aromatic or heteroaromatic ring, optionally substituted.

20. The coating according to any of claim 3, also comprising an oligomeric organic semi-conducting compound comprising elementary units of formula (II-3): 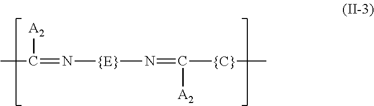
wherein:
the group A₂ represents a group selected from the group consisting in H, F, CN, CF₃, COOR and group {C} represents an aromatic or heteroaromatic cyclic hydrocarbon group, optionally substituted; and
the group {E} represents an aromatic or heteroaromatic ring, optionally substituted.

21. The coating according to any of claim 4, also comprising an oligomeric organic semi-conducting compound comprising elementary units of formula (II-3): 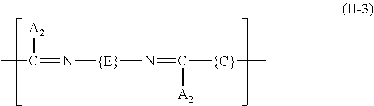
wherein:
the group A₂ represents a group selected from the group consisting in H, F, CN, CF₃, COOR and group {C} represents an aromatic or heteroaromatic cyclic hydrocarbon group, optionally substituted; and
the group {E} represents an aromatic or heteroaromatic ring, optionally substituted.

22. The coating according to claim 2, comprising an organic semi-conducting compound, typically of type N, fitting the formula (I′) and an organic semi-conducting compound typically of type P, fitting the formula (III): 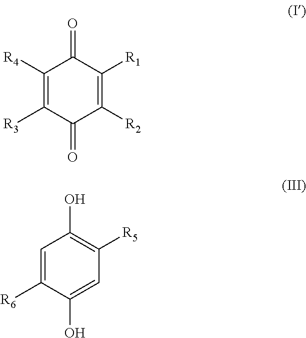
wherein:
each of the groups R₁, R₂, R₃ and R₄ represents independently a hydrogen atom, a halogen atom (F, Cl, Br), a —CN group, or a hydrocarbon group (for example a linear or branched C₁-C₁₂ alkyl group, or an aryl group optionally substituted),
it being understood that two or more of the groups R₁, R₂, R₃ and R₄ may form together an aromatic or heteroaromatic polycyclic structure;
each of the groups R₅ and R₆ represents independently a group H, hydroxyl, alkyl, O-alkyl or aryl,
it being understood that R₅ and/or R₆ may form with the central benzene ring, an aromatic or heteroaromatic structure.

23. The coating according to claim 3, comprising an organic semi-conducting compound, typically of type N, fitting the formula (I′) and an organic semi-conducting compound typically of type P, fitting the formula (III): 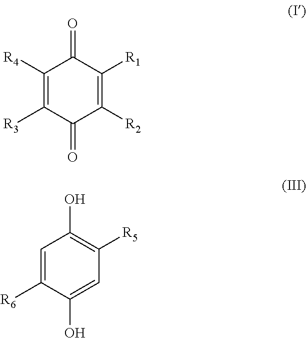
wherein:
each of the groups R₁, R₂, R₃ and R₄ represents independently a hydrogen atom, a halogen atom (F, Cl, Br), a —CN group, or a hydrocarbon group (for example a linear or branched C₁-C₁₂ alkyl group, or an aryl group optionally substituted),
it being understood that two or more of the groups R₁, R₂, R₃ and R₄ may form together an aromatic or heteroaromatic polycyclic structure;
each of the groups R₅ and R₆ represents independently a group H, hydroxyl, alkyl, O-alkyl or aryl,
it being understood that R₅ and/or R₆ may form with the central benzene ring, an aromatic or heteroaromatic structure.
